(19) United States
(12) Patent Application Publication
(10) Pub. No.: US 2020/0368119 A1
(43) Pub. Date: Nov. 26, 2020

(54) OIL-IN-WATER FINE EMULSION COSMETIC COMPOSITION

(71) Applicant: Shiseido Company, Ltd., Chuo-ku, Tokyo (JP)

(72) Inventor: Makoto Uyama, Kanagawa (JP)

(73) Assignee: Shiseido Company, Ltd., Chuo-ku, Tokyo (JP)

(21) Appl. No.: 16/769,815

(22) PCT Filed: Feb. 8, 2019
(86) PCT No.: PCT/JP2019/004734

§ 371 Date: Jun. 4, 2020

(30) Foreign Application Priority Data
Mar. 30, 2018 (JP) 2018-069278

(51) Int. Cl. A61K 8/06; A61K 8/895; A61K 8/86; A61K 8/34; A61Q 19/00

(52) U.S. Cl. CPC A61K 8/062; A61K 8/895; A61K 8/86; A61K 8/342; B82Y 5/00; A61K 2800/21; A61K 2800/413; A61K 2800/5422; A61K 2800/596; A61Q 19/007

(57) ABSTRACT

The invention provides a novel oil-in-water fine emulsion cosmetic composition that is stable.

The oil-in-water fine emulsion cosmetic composition of the invention, containing:
a dispersion medium that contains water and a divalent glycol;
an oil component that is dispersed in the dispersion medium and contains a silicone oil and a hydrocarbon oil;
a carboxy-modified silicone-based surfactant represented by the following Formula 1;
at least one selected from a higher alcohol and a higher fatty acid that are liquid at 25° C. and have 16 to 22 carbon atoms; and
a nonionic surfactant that contains a polyoxyethylene chain and has an HLB of 12 to 17,
the cosmetic composition having oil droplets of an average particle size of 150 nm or smaller:

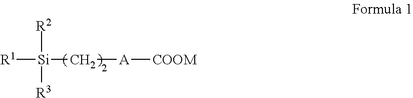

wherein,
at least one of R¹ to R³ is a substituent represented by —O—Si(R⁴)₃ wherein R⁴ is at least one selected from an alkyl group having 1 to 6 carbon atoms and a phenyl group and, when any one of R¹ to R³ is not the substituent, the others are substituted or unsubstituted monovalent hydrocarbon groups that are optionally the same or different;
A is a linear or branched alkylene group represented by C_{q}H_{2q} wherein q is an integer of 0 to 20, and
M is a metal atom or an organic cation.

### FIELD

The present invention relates to a novel oil-in-water fine emulsion cosmetic composition.

### BACKGROUND

In recent years, in the field of cosmetic compositions, oil-in-water fine emulsions and the like have been studied for improvement of, for example, transparency, functionality, and the feeling of use. Such fine emulsions are produced by, for example, mechanically fining emulsified particles in an emulsion using a high-pressure emulsifying apparatus capable of applying a high shear force.

PTL 1 discloses an oil-in-water emulsified composition obtained by fining emulsified particles by high-pressure emulsification, in which composition contains: (A) a salt-type agent; (B) a hydrophilic nonionic surfactant; (C) a monosalt of N-long chain acyl acidic amino acid; (D) two or more kinds of higher fatty acids and an alkali constituting a higher fatty acid soap; (E) a higher alcohol; (F) an oil component; and (G) water.

PTL 2 discloses a fine emulsion-type cosmetic composition which contains: an aqueous phase as a continuous layer; an oil phase that is dispersed in the aqueous phase and contains a silicone oil and a hydrocarbon oil in a total amount of not less than 82% by mass; a specific carboxy-modified silicone; a C16 to 22 higher alcohol; a nonionic surfactant having a POE chain and an HLB of 5 to 10; and a divalent glycol, and in which emulsified particles have an average particle size of 150 nm or smaller. Further, in this PTL 2, it is disclosed to obtain a fine emulsion through a microemulsion without high-pressure emulsification.

### CITATION LIST

### Patent Literature

### [PTL 1] Japanese Unexamined Patent Publication (Kokai) No. 2012-126705

### [PTL 2] Japanese Unexamined Patent Publication (Kokai) No. 2017-066068

### SUMMARY

### Technical Problem

Emulsified particles fined in a cosmetic composition are likely to exhibit a tendency of being relatively unstable; therefore, in the field of cosmetic compositions, a more stable fine emulsion-type cosmetic composition has been desired.

Accordingly, a main object of the present invention is to provide a novel oil-in-water fine emulsion cosmetic composition that is stable. Another main object of the present invention is to provide a method of obtaining the novel oil-in-water fine emulsion cosmetic composition through a microemulsion.

### Solution to Problem

### <Aspect 1>

An oil-in-water fine emulsion cosmetic composition, containing:

a dispersion medium that contains water and a divalent glycol;

an oil component that is dispersed in the dispersion medium and contains a silicone oil and a hydrocarbon oil;

a carboxy-modified silicone-based surfactant represented by the following Formula 1;

at least one selected from a higher alcohol and a higher fatty acid that are liquid at 25° C. and have 16 to 22 carbon atoms; and

a nonionic surfactant that contains a polyoxyethylene chain and has an HLB of 12 to 17,

the cosmetic composition having oil droplets of an average particle size of 150 nm or smaller:

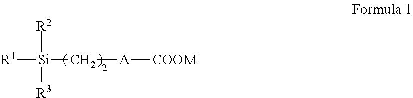

wherein,

at least one of R¹ to R³ is a substituent represented by —O—Si(R⁴)₃ wherein R⁴ is at least one selected from an alkyl group having 1 to 6 carbon atoms and a phenyl group and, when any one of R¹ to R³ is not the substituent, the others are substituted or unsubstituted monovalent hydrocarbon groups that are optionally the same or different;

A is a linear or branched alkylene group represented by C_{q}H_{2q} wherein q is an integer of 0 to 20, and

M is a metal atom or an organic cation.

### <Aspect 2>

The cosmetic composition according to Aspect 1, wherein the carboxy-modified silicone-based surfactant, the at least one selected from the higher alcohol and the higher fatty acid, and the nonionic surfactant form liquid interfacial films of the oil droplets in a 25° C. atmosphere.

### <Aspect 3>

The cosmetic composition according to Aspect 1 or 2, wherein the higher alcohol is at least one selected from isostearyl alcohol and oleyl alcohol, and the higher fatty acid is at least one selected from isostearic acid and oleic acid.

### <Aspect 4>

The cosmetic composition according to any one of Aspects 1 to 3, wherein the silicone oil and the hydrocarbon oil are contained in the oil component in an amount of 82% by mass or more.

### <Aspect 5>

The cosmetic composition according to any one of Aspects 1 to 4, wherein a mass ratio of the silicone oil and the hydrocarbon oil is 1:9 to 9:1.

### <Aspect 6>

The cosmetic composition according to any one of Aspects 1 to 5, wherein a mass ratio of the oil component is 0.45 to 1.0 with respect to a total amount of the carboxy-modified silicone-based surfactant, the at least one selected from the higher alcohol and the higher fatty acid, and the nonionic surfactant.

### <Aspect 7>

The cosmetic composition according to any one of Aspects 1 to 6, wherein the oil droplets have an average particle size of 55 nm or smaller and contain a low alcohol.

### <Aspect 8>

A method of producing the cosmetic composition according to any one of Aspects 1 to 7, the method including:

preparing a microemulsion by mixing a portion of the dispersion medium, the oil component, the carboxy-modified silicone-based surfactant, the at least one selected from the higher alcohol and the higher fatty acid, and the nonionic surfactant; and

diluting the microemulsion with an addition of a remainder of the dispersion medium, or diluting a remainder of the dispersion medium with an addition of the microemulsion.

### <Aspect 9>

The method according to Aspect 8, wherein the remainder of the dispersion medium contains a lower alcohol.

### Advantageous Effects of Invention

According to the present invention, a novel oil-in-water fine emulsion cosmetic composition that is stable, particularly an oil-in-water fine emulsion cosmetic composition that is stable over a prolonged period even when it is maintained at a relatively high temperature of about 40 to 50° C., can be provided.

Further, according to the present invention, a method of obtaining the novel oil-in-water fine emulsion cosmetic composition through a microemulsion, particularly a method of obtaining the fine emulsion cosmetic composition without using a high temperature of, for example, 80° C. or higher, can be provided.

### DESCRIPTION OF EMBODIMENTS

Embodiments of the present invention will now be described in detail. The present invention is, however, not restricted to the below-described embodiments, and can be carried out with various modifications within the scope of the gist of the invention.

The oil-in-water fine emulsion cosmetic composition of the present invention contains: a dispersion medium that contains water and a divalent glycol; an oil component that is dispersed in the dispersion medium and contains a silicone oil and a hydrocarbon oil; a carboxy-modified silicone-based surfactant represented by the following Formula 1; at least one selected from a higher alcohol and a higher fatty acid that are liquid at 25° C. and have 16 to 22 carbon atoms; and a nonionic surfactant that contains a polyoxyethylene chain and has an HLB of 12 to 17, and the oil-in-water fine emulsion cosmetic composition of the present invention has oil droplets of an average particle size of 150 nm or smaller.

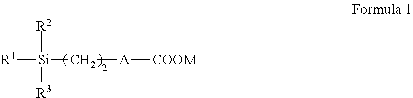

In Formula 1,

at least one of R¹ to R³ is a substituent represented by —O—Si(R⁴)₃ wherein R⁴ is at least one selected from an alkyl group having 1 to 6 carbon atoms and a phenyl group and, when any one of R¹ to R³ is not the substituent, the others are substituted or unsubstituted monovalent hydrocarbon groups that are optionally the same or different;

A is a linear or branched alkylene group represented by C_{q}H_{2q} wherein q is an integer of 0 to 20, and

M is a metal atom or an organic cation.

Although the present invention is not restricted based on a principle, the action and principle that enable to obtain a stable oil-in-water fine emulsion are believed to be as follows.

The present inventor discovered that a microemulsion phase appears at a relatively low temperature when a specific dispersion medium, a specific oil component, two specific surfactants, and a specific higher alcohol or higher fatty acid are used in combination, and that a stable oil-in-water fine emulsion can be prepared by diluting the microemulsion phase with water or the like.

For example, behenyl alcohol used in PTL 2 has a high melting point of 65 to 72° C.; therefore, in an oil-in-water fine emulsion obtained by using this alcohol, it is believed that an interfacial film in the form of a solid or α-gel at 25° C., which is composed of a surfactant and behenyl alcohol, is formed in the interfaces of dispersed oil droplets. On the other hand, in the case of the fine emulsion of the present invention, it is believed that, because of the use of a higher alcohol, a higher fatty acid or the like that is liquid at 25° C., an interfacial film that is liquid at 25° C. is formed on the resulting oil droplets.

Oil droplets having a liquid interfacial film exhibit such a behavior that a surfactant and the like constituting the film replace a surfactant contained in a dispersion medium, or a surfactant and the like of adjacent other oil droplets, in an equilibrium state; therefore, these oil droplets are expected to be less stable as compared to oil droplets having a solid interfacial film that do not exhibit such a behavior. However, in the fine emulsion of the present invention, stable oil droplets are believed to be formed since not only an electrostatic repulsion effect attributed to the carboxy-modified silicone-based surfactant but also a steric repulsion effect attributed to the nonionic surfactant having a specific HLB are exerted in the vicinity of the interfaces of the oil droplets, and aggregation and coalescence of the oil droplets can thereby be further inhibited. Moreover, both of these effects and the difference in the properties of the formed interfacial films are believed to bring about a long-term stability at high temperatures, for example, stability at 40° C. to 50° C. for as long as 30 days.

The terms used in the present invention are defined as follows.

In the present invention, the term “fine emulsion” refers to an oil-in-water type emulsion that contains oil droplets having an average particle size of 150 nm or smaller.

In the present invention, the term “microemulsion” refers to a transparent phase of a water-continuous type (O/W type), a bicontinuous type, or an oil-continuous type (W/O type), which is thermodynamically stable in a system that contains a surfactant, water, an oil component and, depending on the case, an auxiliary agent such as a divalent glycol.

In the present invention, the term “α-gel” refers to a lamellar bimolecular film assembly that contains a hexagonal crystal system as a basic unit, and an α-gel is also called α-type hydrate crystal phase.

### <<Fine Emulsion Cosmetic Composition>>

The cosmetic composition of the present invention is an oil-in-water type emulsified composition in a state where oil droplets as a dispersed phase are finely dispersed in a dispersion medium that is a continuous phase containing water and a divalent glycol.

The cosmetic composition of the present invention can be a transparent or translucent cosmetic composition since the oil droplets are finely dispersed in the dispersion medium. The transparency can be evaluated based on, for example, an L value determined using a color-difference meter such as COLOR-EYE 7000A (manufactured by Gretag Macbeth Holding AG). An L value closer to 100 indicates a higher transparency, and the cosmetic composition of the present invention can have an L value of 80 or higher, 85 or higher, or 90 or higher.

Further, the cosmetic composition of the present invention can provide a long-term stability under high-temperature conditions. For example, when the cosmetic composition is maintained at 40° C. or 50° C. for 30 days, an increase in the average particle size of the oil droplets can be inhibited to be 20% or less, 15% or less, 10% or less, or 5% or less, starting immediately after the preparation of the cosmetic composition.

### <Oil Droplets>

As an oil phase or a dispersed phase in the oil-in-water type emulsified composition, the oil droplets may contain an oil component, a carboxy-modified silicone-based surfactant, a nonionic surfactant, and at least one selected from a higher alcohol and a higher fatty acid.

From the standpoints of the fine emulsion formability and the like, the mass ratio of the oil component can be set at 0.45 or higher, 0.50 or higher, or 0.55 or higher, and 1.0 or lower, 0.95 or lower, or 0.90 or lower, with respect to a total amount of the carboxy-modified silicone-based surfactant, the at least one selected from the higher alcohol and the higher fatty acid, and the nonionic surfactant.

For example, from the standpoint of obtaining a transparent or translucent emulsified composition, the average particle size of the oil droplets in the cosmetic composition of the present invention can be 150 nm or smaller, 140 nm or smaller, 130 nm or smaller, 120 nm or smaller, or 110 nm or smaller, and the average particle size is preferably 100 nm or smaller, 80 nm or smaller, or 60 nm or smaller. A lower limit value of the average particle size is not particularly restricted, and it may be 5 nm or larger, 10 nm or larger, or 15 nm or larger. The average particle size of the oil droplets can be defined as, for example, an average diameter value of the oil droplets optically measured by a dynamic light scattering method or the like, assuming that the particle shape of the oil droplets is spherical.

### (Oil Component)

The oil component used in the cosmetic composition of the present invention contains at least two kinds of oils, which are a silicone oil and a hydrocarbon oil. Generally, the use of a combination of a silicone oil and a hydrocarbon oil in a cosmetic composition often leads to separation of these oils; however, in the cosmetic composition of the present invention, because of the use of two specific surfactants, at least two kinds of oils that are a silicone oil and a hydrocarbon oil can be incorporated without separation thereof.

Examples of the silicone oil that can be used include, but not limited to: linear silicones, such as dimethylpolysiloxane (dimethicone), methylphenylpolysiloxane, and methyl hydrogen polysiloxane; and cyclic silicones, such as octamethylcyclotetrasiloxane, decamethylcyclopentasiloxane, and dodecamethylcyclohexasiloxane.

Examples of the hydrocarbon oil that can be used include, but not limited to: liquid paraffin, squalane, squalene, paraffin, isoparaffin, ceresin, hydrogenated polydecene, isododecane, and isohexadecane.

The oil component may also contain other oil such as a polar oil in addition to the silicone oil and the hydrocarbon oil; however, from standpoints of the fine emulsion formability and the like, it is preferred that the silicone oil and the hydrocarbon oil be contained in the oil component in an amount of 82% by mass or more, 85% by mass or more, or 90% by mass or more, and it is more preferred that the oil component consist of only the silicone oil and the hydrocarbon oil. Further, from standpoints of the fine emulsion formability and the like, the mass ratio of the silicone oil and the hydrocarbon oil is preferably 1:9 to 9:1, more preferably 2:8 to 8:2, particularly preferably 3:7 to 7:3.

The content of the oil component in the cosmetic composition of the present invention can be, for example, but not limited to: 0.04% by mass or more, 0.07% by mass or more, or 0.10% by mass or more, and 10% by mass or less, 8% by mass or less, or 6% by mass or less, with respect to a total amount of the cosmetic composition.

### (Carboxy-Modified Silicone-Based Surfactant)

The carboxy-modified silicone-based surfactant used in the cosmetic composition of the present invention is a surfactant that is a carboxy-modified silicone modified with an alkylcarboxyl group, which is a compound represented by the following Formula 1.

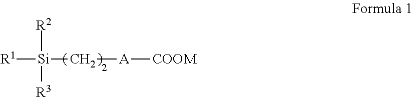

In Formula 1, at least one of R¹ to R³ is a substituent represented by —O—Si(R⁴)₃ wherein R⁴ is at least one selected from an alkyl group having 1 to 6 carbon atoms and a phenyl group and, when any one of R¹ to R³ is not the substituent, the others are substituted or unsubstituted monovalent hydrocarbon groups that are optionally the same or different; A is a linear or branched alkylene group represented by C_{q}H_{2q} wherein q is an integer of 0 to 20, and M is a metal atom or an organic cation.

In the substituent represented by —O—Si(R⁴)₃, R⁴ is at least one selected from an alkyl group having 1 to 6 carbon atoms and a phenyl group, preferably an alkyl group having 1 to 6 carbon atoms. Examples of the alkyl group having 1 to 6 carbon atoms include linear, branched or cyclic alkyl groups, such as methyl, ethyl, n-propyl, i-propyl, n-butyl, i-butyl, s-butyl, t-butyl, pentyl, neopentyl, cyclopentyl, and hexyl. Specific examples of the substituent represented by —O—Si(R⁴)₃ include —O—Si(CH₃)₃, —O—Si(CH₃)₂(C₂H₅), —O—Si(CH₃)₂(C₃H₇), —O—Si(CH₃)₂(C₄H₉), —O—Si(CH₃)₂(C₅H₁₁), —O—Si(CH₃)₂(C₆H₁₃), and —O—Si(CH₃)₂(C₆H₅), among which a trialkylsiloxy group is preferred, and a trimethylsiloxy group is more preferred.

In Formula 1, at least one of R¹ to R³ may be the substituent represented by —O—Si(R⁴)₃ and, when any one of R¹ to R³ is not the substituent, the others may be substituted or unsubstituted monovalent hydrocarbon groups that are optionally the same or different.

Examples of the unsubstituted monovalent hydrocarbon groups include: linear, branched or cyclic alkyl groups, such as methyl, ethyl, n-propyl, i-propyl, n-butyl, i-butyl, s-butyl, pentyl, neopentyl, cyclopentyl, and hexyl; aryl groups, such as a phenyl group, a tolyl group, or a xylyl group; and aralkyl groups.

Examples of the substituted monovalent hydrocarbon groups include: perfluoroalkyl groups, such as a 3,3,3-trifluoropropyl group and a 3,3,4,4,4-pentafluorobutyl group; aminoalkyl groups, such as a 3-aminopropyl group and a 3-(2-aminoethylamino)propyl group; and amide alkyl groups, such as an acetylaminoalkyl group.

The substituted or unsubstituted monovalent hydrocarbon groups may be partially substituted with a hydroxy group, an alkoxy group, a polyether group, or a perfluoropolyether group, and examples of the alkoxy group include a methoxy group, an ethoxy group, and a propoxy group.

In Formula 1, when one or two of R¹ to R³ is/are a substituent(s) represented by —O—Si(R⁴)₃, the other(s) is/are preferably a linear or branched alkyl group(s) having 1 to 6 carbon atoms, more preferably a methyl group(s) or an ethyl group(s). Particularly, two or all of R¹ to R³ are preferably substituents represented by —O—Si(R⁴)₃ and, when two of R¹ to R³, for example, R¹ and R² are such substituents, the remaining R³ is preferably a linear or branched alkyl group having 1 to 6 carbon atoms, more preferably a methyl group or an ethyl group.

In Formula 1, A is a linear or branched alkylene group represented by C_{q}H_{2q}, and q is an integer of 0 to 20. When q=0, the carboxy-modified silicone-based surfactant represented by Formula 1 is a compound represented by the following Formula 2, and its carboxyl-modified group is bound with silicon via an ethylene group. Further, q is preferably an integer of 6 to 20, more preferably an integer of 2 to 15, particularly preferably an integer of 6 to 12.

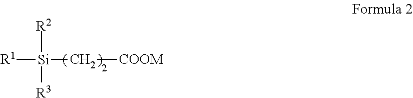

In Formula 1, M is a metal atom or an organic cation. The metal atom is, for example, a monovalent alkali metal or a divalent alkaline earth metal. Examples of the monovalent alkali metal include Li, Na and K, and examples of the divalent alkaline earth metal include Mg, Ca, and Ba. Other examples of the metal atom include Mn, Fe, Co, Al, Ni, Cu, V, Mo, Nb, Zn, and Ti. Further, examples of the organic cation include an ammonium ion, a monoethanol ammonium ion, a triethanol ammonium ion, an arginine-neutralized ion, and an aminomethyl propanol-neutralized ion. M is particularly preferably a monovalent alkali metal or a monovalent organic cation.

In the preparation of the cosmetic composition of the present invention, the carboxy-modified silicone-based surfactant may be used in a state of being neutralized as illustrated in Formula 1, or raw materials of the surfactant represented by Formula 1 wherein M is a hydrogen atom may be mixed with a neutralizer and neutralized during the preparation of the cosmetic composition.

Examples of the neutralizer include, but not limited to: potassium hydroxide, sodium hydroxide, monoethanolamine, diethanolamine, triethanolamine, 2-amino-2-methylpropanol, 2-amino-2-methyl-1,3-propanediol, N-methyl taurine salts, triethylamine, and tributylamine, among which potassium hydroxide, sodium hydroxide, triethanolamine, and 2-amino-2-methyl-1,3-propanediol are preferred.

The content of the carboxy-modified silicone-based surfactant in the cosmetic composition of the present invention can be, for example, but not limited to: 0.05% by mass or more, 0.07% by mass or more, or 0.10% by mass or more, and 2.0% by mass or less, 1.5% by mass or less, or 1.0% by mass or less, with respect to a total amount of the cosmetic composition.

(Higher Alcohol and Higher Fatty Acid that are Liquid at 25° C. and have 16 to 22 Carbon Atoms)

The higher alcohol and the higher fatty acid that are used in the cosmetic composition of the present invention may be any higher alcohol and any higher fatty acid as long as they are each liquid at 25° C. and have 16 to 22 carbon atoms. As the higher alcohol and the higher fatty acid, one or more kinds of higher alcohols and higher fatty acids can be used, respectively, and a higher alcohol and a higher fatty acid may be used in combination as well. The higher alcohol and the higher fatty acid are liquid at 25° C.; therefore, they do not require a heating treatment performed at a high temperature of 80° C. and are capable of forming a microemulsion phase in the vicinity of a room temperature of, for example, about 25 to 40° C.

A total content of the higher alcohol and the higher fatty acid in the cosmetic composition of the present invention can be, for example, but not limited to: 0.05% by mass or more, 0.07% by mass or more, or 0.10% by mass or more, and 2.0% by mass or less, 1.5% by mass or less, or 1.0% by mass or less, with respect to a total amount of the cosmetic composition. The term “total content” used herein refers to a total amount of the higher alcohol and the higher fatty acid when they are both used in the cosmetic composition, or the amount of either the higher alcohol or the higher fatty acid when only one of them is used in the cosmetic composition.

Examples of the higher alcohol include, but not limited to: oleyl alcohol, isostearyl alcohol, octyl dodecanol, decyl tetradecanol, and Jojoba alcohol. Thereamong, from the standpoints of the ease of forming a microemulsion phase and the like, oleyl alcohol and isostearyl alcohol are preferred, and isostearyl alcohol is more preferred.

Examples of the higher fatty acid include, but not limited to: oleic acid, isostearic acid, linoleic acid, linolenic acid, eicosapentaenoic acid, and docosahexaenoic acid. Thereamong, from the standpoints of the ease of forming a microemulsion phase and the like, oleic acid and isostearic acid are preferred, and isostearic acid is more preferred.

(Nonionic Surfactant that Contains Polyoxyethylene Chain and has HLB of 12 to 17)

The nonionic surfactant used in the cosmetic composition of the present invention may be any nonionic surfactant as long as it contains a polyoxyethylene chain and has an HLB of 12 to 17. From the standpoints of steric repulsion and emulsification stability, the average addition molar number of polyoxyethylene chain is preferably 10 to 100, more preferably 15 to 30, and the HLB is preferably 12 to 14. The HLB, which is generally a value that indicates the affinity of a surfactant to water and oils, is a parameter known as hydrophilicity-lipophilicity balance and can be easily determined by a known calculation method, such as the Griffin method.

Examples of the nonionic surfactant include, but not limited to: polyoxyethylene alkyl ether, polyethylene glycol fatty acid ester, polyoxyethylene hardened castor oil, and polyoxyethylene fatty acid glyceryl, and these nonionic surfactants may be used singly, or in combination of two or more thereof. Further, from the standpoint of, for example, the ease of forming a microemulsion phase in the vicinity of room temperature, a branched nonionic surfactant that is likely to be liquid in the vicinity of room temperature is preferred.

Specific examples of the nonionic surfactant include oleth-15 of POE(15) oleyl ether (HLB=12), PEG-15 glyceryl isostearate (HLB=12), PEG-15 glyceryl stearate (HLB=13), PEG-20 glyceryl isostearate (HLB=13), PEG-7 glyceryl cocoate (HLB=13), PEG-20 glyceryl stearate (HLB=14), isostealeth-25 of POE(25) isostearyl ether (HLB=14), PEG-30 glyceryl isostearate (HLB=15), POE(20) behenyl ether (HLB=16.5), and PEG-100 stearate (HLB=17). Thereamong, POE(15) oleyl ether (HLB=12), PEG-20 glyceryl isostearate (HLB=13), POE(25) isostearyl ether (HLB=14), PEG-30 glyceryl isostearate (HLB=15), and PEG-100 stearate (HLB=17) are preferred, and POE(15) oleyl ether (HLB=12), PEG-20 glyceryl isostearate (HLB=13), and POE(25) isostearyl ether (HLB=14) are more preferred. It is noted here that “POE” and “PEG” denote polyoxyethylene and polyethylene glycol, respectively.

The content of the above-described nonionic surfactants in the cosmetic composition of the present invention can be, for example, but not limited to: 0.05% by mass or more, 0.07% by mass or more, or 0.10% by mass or more, and 2.0% by mass or less, 1.5% by mass or less, or 1.0% by mass or less, with respect to a total amount of the cosmetic composition.

### <Dispersion Medium>

### (Water)

The water used as the dispersion medium is not particularly restricted and may be, for example, distilled water, purified water, or ion exchanged water. The content of the water may be adjusted as appropriate in accordance with the use and the like of the cosmetic composition and can be, for example, but not particularly limited to: 70% by mass or more, 75% by mass or more, or 80% by mass or more, and 99% by mass or less, 98% by mass or less, or 97% by mass or less, with respect to a total amount of the cosmetic composition.

### (Divalent Glycol)

In the present invention, a divalent glycol is used as a dispersion medium for the formation of a microemulsion phase. The divalent glycol is not particularly restricted as long as a microemulsion phase can be formed and, for example, dipropylene glycol, ethylene glycol, diethylene glycol, propylene glycol, and 1,3-butylene glycol may be used singly, or in combination of two or more thereof. The content of the divalent glycol can be, for example, but not particularly limited to: 0.1% by mass or more, 0.2% by mass or more, or 0.3% by mass or more, and 5% by mass or less, 4% by mass or less, or 3% by mass or less, with respect to a total amount of the cosmetic composition.

### (Alkylene Oxide Derivative)

In the present invention, an alkylene oxide derivative can be used in combination with the divalent glycol. The alkylene oxide derivative can be used as a dispersion medium in the same manner as the divalent glycol, and is capable of exerting an effect of assisting fine emulsification for the nonionic surfactant. Examples of the alkylene oxide derivative include compounds represented by the following Formula 3.

R⁵O-[(AO)ᵣ(EO)ₛ]-R⁶ Formula 3

In Formula 3, AO is oxyalkylene group having 3 to 4 carbon atoms, and EO is an oxyethylene group. Specific examples of AO include an oxypropylene group, an oxybutylene group, an oxyisobutylene group, an oxytrimethylene group, and an oxytetramethylene group, among which an oxypropylene group and an oxybutylene group are preferred.

The ratio of AO with respect to a total amount of AO and EO is preferably 20 to 80% by mass, more preferably 30 to 70% by mass.

An order of adding AO and EO is not particularly specified, and AO and EO may be added in a block form or a random form; however, AO and EO are preferably added in a random form. The block form encompasses not only a two-stage block but also a three- or more-step block.

In Formula 3, r and s are average addition molar numbers of oxyalkylene group and added oxyethylene group, respectively, and preferably satisfy 1≤r≤70 and 1≤s≤70, or 2≤r≤60 and 2≤s≤60. Further, from the standpoints of stickiness and the like, a total of r and s is preferably 100 or less.

R⁵ and R⁶ are each an alkyl group having 1 to 4 carbon atoms, and examples of the alkyl group include a methyl group, an ethyl group, an n-propyl group, an isopropyl group, an n-butyl group, a sec-butyl group, and a tert-butyl group, among which a methyl group and an ethyl group are preferred. R⁵ and R⁶ may be the same or different.

The alkylene oxide derivative can be produced by a known method, for example, by addition-polymerizing ethylene oxide and an alkylene oxide having of 3 to 4 carbon atoms to a hydroxy group-containing compound and subsequently performing an etherification reaction of a halogenated alkyl in the presence of an alkaline catalyst.

Specific examples of the alkylene oxide derivative represented by Formula 3 include, but not limited to: POE(17) POE(4) dimethyl ether, POE(14) POP(7) dimethyl ether, POE(36) POP(41) dimethyl ether, POE(55) POP(28) dimethyl ether, POE(22) POP(40) dimethyl ether, POE(35) POP(40) dimethyl ether, POE(50) POP(40) dimethyl ether, and POE(11) POP(9) dimethyl ether.

When the alkylene oxide derivative is used in combination, a total content of the divalent glycol and the alkylene oxide derivative can be, for example, 0.1% by mass or more, 0.2% by mass or more, or 0.3% by mass or more, and 25% by mass or less, 20% by mass or less, or 15% by mass or less, with respect to a total amount of the cosmetic composition. Further, a mass ratio of the divalent glycol and the alkylene oxide derivative is preferably 3:7 to 8:2, more preferably 4:6 to 8:2, particularly preferably 5:5 to 8:2.

### (Optional Components)

In the cosmetic composition of the present invention, various components may be incorporated as appropriate within a range that does not affect the effects of the present invention. Examples of the various components include additive components that can be normally incorporated into cosmetic compositions, for example, oil components other than the one described above, such as liquid fats, solid fats, and waxes; higher alcohols other than the one described above; higher fatty acids other than the one described above; anionic surfactants other than the one described above; cationic surfactants; amphoteric surfactants; nonionic surfactants other than the one described above; moisturizers; water-soluble polymers; thickeners; film-forming agents, such as silicone-modified polysaccharides; metal ion sequestering agents; lower alcohols; polyhydric alcohols; various extracts; sugars; amino acids; organic amines; polymer emulsions; chelating agents; UV absorbers; pH modifiers; skin nutrients; vitamins; water-soluble drug agents that can be applied to pharmaceuticals, quasi drugs, cosmetics and the like; antioxidants; buffers; preservatives; antioxidant aids; propellants; organic powders; pigments; dyes; color agents; and fragrance agents.

### (Use Application)

The use application of the cosmetic composition of the present invention is not particularly restricted, and the cosmetic composition of the present invention is preferably used in, for example, skin lotions and beauty lotions because of its excellent stability and the like. In addition, the cosmetic composition of the present invention can be utilized in various cosmetics, examples of which include: skin care cosmetics, such as moisturizing creams, moisturizing milky lotions, moisturizing lotions, massage creams, massage lotions, and essence; hair care cosmetics, such as hair creams, hair lotions, and hair dressings; body care cosmetics, such as sunscreens, body creams, and body lotions; makeup cosmetics, such as lipsticks, mascaras, eye liners, nail enamels, liquid foundations, and gel foundations; and cleansers, such as makeup removers, shampoos, hair conditioners, and two-in-one shampoos.

### <<Method of Producing Fine Emulsion Cosmetic Composition>

The fine emulsion cosmetic composition of the present invention can be prepared through a microemulsion phase; therefore, it is not necessary to use a high-pressure emulsifying apparatus. Specifically, the fine emulsion cosmetic composition of the present invention can be produced by, for example, mixing a portion of a dispersion medium containing water and a divalent glycol, an oil component, a carboxy-modified silicone-based surfactant, at least one selected from a higher alcohol and a higher fatty acid, and a nonionic surfactant to prepare a microemulsion, and subsequently diluting the microemulsion with an addition of the remainder of the dispersion medium, or diluting the remainder of the dispersion medium with an addition of the microemulsion.

The carboxy-modified silicone-based surfactant may be used in a state of being neutralized as illustrated in Formula 1, or raw materials of the surfactant represented by Formula 1 wherein M is a hydrogen atom may be mixed with a neutralizer and neutralized during the preparation of the cosmetic composition. Further, the remainder of the dispersion medium can be divided and used in two or more fractions. In this case, a fraction of the remainder of the dispersion medium that is used first contains water and may optionally further contain a divalent glycol and a lower alcohol and, when this first fraction of the remainder of the dispersion medium does not contain a divalent glycol, the rest of the fractions of the remainder of the dispersion medium contains water and divalent glycol, whereas when the first fraction of the remainder of the dispersion medium contains a divalent glycol, the rest of the fractions of the remainder of the dispersion medium contain water and optionally a divalent glycol. In either of these cases, the above-described optional components may be incorporated as appropriate.

In the present invention, a microemulsion phase-forming temperature can be set at 10° C. or higher, 15° C. or higher, or 20° C. or higher, and 75° C. or lower, 70° C. or lower, or 65° C. or lower. From the standpoints of the productivity and the like, the microemulsion phase-forming temperature is preferably in a range of 25° C. to 50° C., more preferably in a range of 25° C. to 45° C. The cosmetic composition of the present invention is prepared by forming a microemulsion phase at such a temperature and subsequently diluting the microemulsion phase with a dispersion medium; therefore, it is not necessary to apply a heating treatment at a high temperature of 50° C. or higher, 55° C. or higher, 60° C. or higher, 65° C. or higher, 70° C. or higher, 75° C. or higher, or 80° C. or higher, which is conventionally performed in the preparation of a fine emulsion.

Further, it is preferred to incorporate a lower alcohol into the remainder of the dispersion medium that is used for diluting the microemulsion. By the use of a lower alcohol, the average particle size of oil droplets in the resulting cosmetic composition can be further reduced. This lower alcohol is preferably a monohydric alcohol that contains an alkyl group having 1 to 5 carbon atoms, more preferably a monohydric alcohol that contains an alkyl group having 1 to 3 carbon atoms. Moreover, the content of the lower alcohol to be incorporated into the remainder of the dispersion medium can be 35% by mass or less, 30% by mass or less, or 25% by mass or less, with respect to a total amount of the remainder of the dispersion medium.

An effect of reducing the average particle size of oil droplets that is exerted by the lower alcohol cannot be obtained when the lower alcohol is separately added to a cosmetic composition after the preparation of the cosmetic composition by dilution of a microemulsion with water. Therefore, even in the case of a system in which the lower alcohol is contained in a cosmetic composition, whether or not the cosmetic composition is a product obtained by the production method of the present invention can be sufficiently distinguished on the basis of whether or not the average particle size of oil droplets in the cosmetic composition is 55 nm or smaller, 50 nm or smaller, or 45 nm or smaller.

### EXAMPLES

The present invention will now be described in more detail by way of Examples thereof; however, the present invention is not restricted to the below-described Examples. Hereinafter, unless otherwise specified, all amounts are indicated in parts by mass.

### Examples 1 to 30 and Comparative Examples 1 to 8

For each of the cosmetic compositions of the present invention that were obtained by the respective formations and production methods indicated in Tables 1 to 9 below, the average particle size of oil droplets contained therein was evaluated. It is noted here that the term “Remainder of dispersion medium for dilution” used in these tables refers to, in a dispersion medium ultimately incorporated into the respective cosmetic compositions, a portion of the dispersion medium that remained after being used for the preparation of a microemulsion phase and was used for diluting the microemulsion phase to prepare the respective cosmetic compositions. The average particle size of oil droplets was directly measured using ZETASIZER Nano ZS (manufactured by Sysmex Corporation), without diluting each sample. The average particle size of oil droplets serves as an index for evaluating the stability of each cosmetic composition, and it can be said that a cosmetic composition containing oil droplets of larger than 150 nm in average particle size is likely to be cloudy due to aggregation and coalescence of the oil droplets and is thus unstable.

### <Effects of Nonionic Surfactants>

The effects of nonionic surfactants on cosmetic compositions were examined based on Examples 1 to 5 and Comparative Examples 1 to 4.

TABLE 1

| | | Compar- | Compar- | Compar- | Compar- | | | | | |
|---|---|---|---|---|---|---|---|---|---|---|
| | | ative | ative | ative | ative | | | | | |
| | | Example 1 | Example 2 | Example 3 | Example 4 | Example 1 | Example 2 | Example 3 | Example 4 | Example 5 |
| Microemulsion | Carboxydecyltrisiloxane | 0.2 | 0.2 | 0.2 | 0.2 | 0.2 | 0.2 | 0.2 | 0.2 | 0.2 |
| phase | Isostearyl alcohol | 0.3 | 0.3 | 0.3 | 0.3 | 0.3 | 0.3 | 0.3 | 0.3 | 0.3 |
| | PEG-5 glyceryl | 0.4 | | | | | | | | |
| | stearate (HLB = 8) | | | | | | | | | |
| | PEG-5 glyceryl | | 0.4 | | | | | | | |
| | isostearate (HLB = 8) | | | | | | | | | |
| | PEG-8 glyceryl | | | 0.4 | | | | | | |
| | isostearate (HLB = 10) | | | | | | | | | |
| | Isosteareth-10 | | | | 0.4 | | | | | |
| | (HLB = 11) | | | | | | | | | |
| | Oleth-15 (HLB = 12) | | | | | 0.4 | | | | |
| | PEG-20 glyceryl | | | | | | 0.4 | | | |
| | isostearate (HLB = 13) | | | | | | | | | |
| | Isosteareth-25 | | | | | | | 0.4 | | |
| | (HLB = 14) | | | | | | | | | |
| | PEG-30 glyceryl | | | | | | | | 0.4 | |
| | isostearate (HLB = 15) | | | | | | | | | |
| | PEG-100 stearate | | | | | | | | | 0.4 |
| | (HLB = 17) | | | | | | | | | |
| | Dimethylpolysiloxane | 0.35 | 0.35 | 0.35 | 0.35 | 0.35 | 0.35 | 0.35 | 0.35 | 0.35 |
| | Hydrogenated | 0.35 | 0.35 | 0.35 | 0.35 | 0.35 | 0.35 | 0.35 | 0.35 | 0.35 |
| | polydecene | | | | | | | | | |
| | Dipropylene glycol | 0.6 | 0.6 | 0.6 | 0.6 | 0.6 | 0.6 | 0.6 | 0.6 | 0.6 |
| | POE(14) POP(7) | 0.4 | 0.4 | 0.4 | 0.4 | 0.4 | 0.4 | 0.4 | 0.4 | 0.4 |
| | dimethyl ether | | | | | | | | | |
| | Ion exchanged water | 1 | 1 | 1 | 1 | 1 | 1 | 1 | 1 | 1 |
| | Aminomethyl | 0.05 | 0.05 | 0.05 | 0.05 | 0.05 | 0.05 | 0.05 | 0.05 | 0.05 |
| | propanediol | | | | | | | | | |
| Remainder of | Ion exchanged water | 96.35 | 96.35 | 96.35 | 96.35 | 96.35 | 96.35 | 96.35 | 96.35 | 96.35 |
| dispersion | | | | | | | | | | |
| medium for | | | | | | | | | | |
| dilution | | | | | | | | | | |

| | | | | | | | | | |
|---|---|---|---|---|---|---|---|---|---|
| Microemulsion phase- | 69 to 82 | 70 to 85 | 41 to 74 | 34 to 66 | 44 to 72 | 27 to 43 | 43 to 63 | 42 to 60 | 42 to 52 |
| forming temperature (° C.) | | | | | | | | | |
| Average particle size (nm) | 630 | 510 | 370 | 210 | 91 | 83 | 63 | 110 | 103 |

### Example 1

Carboxydecyltrisiloxane which is a raw material of a carboxy-modified silicone-based surfactant, aminomethyl propanediol which is a neutralizer of carboxydecyltrisiloxane, isostearyl alcohol, oleth-15 which is a nonionic surfactant, dimethylpolysiloxane and hydrogenated polydecene used as oil components, POE(14) POP(7) dimethyl ether, and ion exchanged water were dissolved by mixing at 45° C. to form a microemulsion. Subsequently, the thus formed microemulsion was added to ion exchanged water, which was a remainder of a dispersion medium and used for dilution in the preparation of a cosmetic composition, and the resultant was mixed with stirring, whereby a cosmetic composition of Example 1 was prepared.

### Examples 2 to 5

Cosmetic compositions of Examples 2 to 5 were each prepared in the same manner as in Example 1, except that the nonionic surfactant was changed to PEG-20 glyceryl isostearate or the like as indicated in Table 1, and that the microemulsion phase-forming temperature was changed to 30° C. in Example 2.

### Comparative Examples 1 to 4

Cosmetic compositions of Comparative Examples 1 to 4 were each prepared in the same manner as in Example 1, except that the nonionic surfactant was changed to PEG-5 glyceryl stearate or the like as indicated in Table 1, and that the microemulsion phase-forming temperature was changed to 75° C. in Comparative Examples 1 and 2.

### (Results)

As apparent from Table 1, it was confirmed that, in the cosmetic compositions of Examples 1 to 5 in which a polyoxyethylene chain-containing nonionic surfactant having an HLB of 12 to 17 was used, oil droplets had a small average particle size of 110 nm or smaller and were stably dispersed in the dispersion medium, while in the cosmetic compositions of Comparative Examples 1 to 4 in which a nonionic surfactant having an HLB of less than 12 was used, oil droplets had a large average particle size of 210 nm or larger, making the cosmetic compositions cloudy and unstable.

### <Effects of Higher Alcohols and Higher Fatty Acids>

The effects of higher alcohols and higher fatty acids on cosmetic compositions were examined based on Examples 2 and 6 to 8 as well as Comparative Example 5.

TABLE 2

| | | Compar- | | | | |
|---|---|---|---|---|---|---|
| | | ative | | | | |
| | | Example 5 | Example 2 | Example 6 | Example 7 | Example 8 |
| Microemulsion | Carboxydecyltrisiloxane | 0.2 | 0.2 | 0.2 | 0.2 | 0.2 |
| phase | Stearyl alcohol | 0.3 | | | | |
| | Isostearyl alcohol | | 0.3 | | | |
| | Oleyl alcohol | | | 0.3 | | |
| | Isostearic acid | | | | 0.3 | |
| | Oleic acid | | | | | 0.3 |
| | PEG-20 glyceryl | 0.4 | 0.4 | 0.4 | 0.4 | 0.4 |
| | isostearate (HLB = 13) | | | | | |
| | Dimethylpolysiloxane | 0.35 | 0.35 | 0.35 | 0.35 | 0.35 |
| | Hydrogenated polydecene | 0.35 | 0.35 | 0.35 | 0.35 | 0.35 |
| | Dipropylene glycol | 0.6 | 0.6 | 0.6 | 0.6 | 0.6 |
| | POE(14) POP(7) | 0.4 | 0.4 | 0.4 | 0.4 | 0.4 |
| | dimethyl ether | | | | | |
| | Ion exchanged water | 1 | 1 | 1 | 1 | 1 |
| | Aminomethyl | 0.05 | 0.05 | 0.05 | 0.05 | 0.05 |
| | propanediol | | | | | |
| Remainder of | Ion exchanged water | 96.35 | 96.35 | 96.35 | 96.35 | 96.35 |
| dispersion | | | | | | |
| medium for | | | | | | |
| dilution | | | | | | |

| | | | | | |
|---|---|---|---|---|---|
| Microemulsion phase- | 50 to 81 | 27 to 43 | 37 to 70 | 33 to 70 | 40 to 72 |
| forming temperature (° C.) | | | | | |
| Average particle size (nm) | — | 83 | 134 | 84 | 121 |

### Examples 6 to 8

Cosmetic compositions of Examples 6 to 8 were each prepared in the same manner as in Example 2, except that isostearyl alcohol was changed to oleyl alcohol in Example 6, isostearic acid in Example 7, or oleic acid in Example 8, and that the microemulsion phase-forming temperature was changed to 45° C. in Examples 6 to 8.

### Comparative Example 5

A cosmetic composition of Comparative Example 5 was prepared in the same manner as in Example 2, except that isostearyl alcohol was changed to stearyl alcohol, and that the microemulsion phase-forming temperature was changed to 60° C.

### (Results)

As apparent from Table 2, it was confirmed that, in the cosmetic compositions of Examples 2 and 6 to 8 in which a higher alcohol or a higher fatty acid that was liquid at 25° C. was used, oil droplets had a small average particle size of 134 nm or smaller and were stably dispersed in the dispersion medium, while in the cosmetic composition of Comparative Example 5 in which stearyl alcohol that is a higher alcohol being solid at 25° C. was used, crystals were precipitated, making the cosmetic composition unstable to such an extent that it was impossible to measure the average particle size.

### <Long-Term Stability Under High Temperatures>

The long-term stability of cosmetic compositions under high temperatures was examined based on Example 9 and Comparative Example 6.

TABLE 3

| | | | Comparative |
|---|---|---|---|
| | | Example 9 | Example 6 |
| Microemulsion phase | Carboxydecyltrisiloxane | 0.2 | 0.6 |
| | Isostearyl alcohol | 0.3 | |
| | Cetyl alcohol | | 0.66 |
| | PEG-20 glyceryl isostearate (HLB = 13) | 0.4 | |
| | PEG-5 glyceryl stearate (HLB = 8) | | 1.28 |
| | Dimethylpolysiloxane | 0.35 | 1.8 |
| | Hydrogenated polydecene | 0.35 | 1.8 |
| | Dipropylene glycol | 1 | 3.6 |
| | Ion exchanged water | 1 | 4.68 |
| | Aminomethyl propanediol | 0.05 | |
| | Triethanolamine | | 0.18 |
| Remainder of dispersion | Ion exchanged water | 96.35 | 85.4 |
| medium for dilution | | | |
| Average particle size (nm) | Immediately after preparation | 87 | 107 |
| | 25° C., after 30 days | 85 | 114 |
| | 40° C., after 30 days | 88 | 141 |
| | 50° C., after 30 days | 90 | 162 |

### Example 9

A cosmetic composition of Example 9 was prepared in the same manner as in Example 2, except that POE(14) POP(7) dimethyl ether was not used.

### Comparative Example 6

Carboxydecyltrisiloxane which is a raw material of a carboxy-modified silicone-based surfactant, triethanolamine which is a neutralizer of carboxydecyltrisiloxane, cetyl alcohol, PEG-5 glyceryl stearate which is a nonionic surfactant, dimethylpolysiloxane and hydrogenated polydecene used as oil components, and ion exchanged water were dissolved by mixing at 80° C. to form a microemulsion. Subsequently, the thus formed microemulsion was added to ion exchanged water, which was a remainder of a dispersion medium and used for dilution, and the resultant was mixed with stirring, whereby a cosmetic composition of Comparative Example 6 was prepared. It is noted here that this cosmetic composition of Comparative Example 6 corresponds to the constitution of PTL 2.

### (Results)

As apparent from Table 3, the cosmetic composition of Comparative Example 6 was observed with not much of an increase in average particle size and is thus considered relatively stable immediately after the preparation or under a temperature condition of about 25° C.; however, this cosmetic composition was confirmed to lack long-term stability under high temperatures, exhibiting an increase in average particle size of about 30 to 50% at temperatures of about 40° C. to 50° C. as compared to immediately after the preparation. On the other hand, the cosmetic composition of Example 9 was confirmed to have excellent long-term stability under high temperatures, exhibiting an increase in average particle size of only about 1 to 3% even at temperatures of about 40° C. to 50° C.

### <Effects of Oil Components>

The effects of oil components on cosmetic compositions were examined based on Examples 9 to 13.

TABLE 4

| | | Example 10 | Example 11 | Example 9 | Example 12 | Example 13 |
|---|---|---|---|---|---|---|
| Microemulsion | Carboxydecyltrisiloxane | 0.2 | 0.2 | 0.2 | 0.2 | 0.2 |
| phase | Isostearyl alcohol | 0.3 | 0.3 | 0.3 | 0.3 | 0.3 |
| | PEG-20 glyceryl | 0.4 | 0.4 | 0.4 | 0.4 | 0.4 |
| | isostearate (HLB = 13) | | | | | |
| | Dimethylpolysiloxane | 0.45 | 0.4 | 0.35 | 0.3 | 0.2 |
| | Hydrogenated | 0.45 | 0.4 | 0.35 | 0.3 | 0.2 |
| | polydecene | | | | | |
| | Dipropylene glycol | 1 | 1 | 1 | 1 | 1 |
| | Ion exchanged water | 1 | 1 | 1 | 1 | 1 |
| | Aminomethyl | 0.05 | 0.05 | 0.05 | 0.05 | 0.05 |
| | propanediol | | | | | |
| Remainder of | Ion exchanged water | 96.15 | 96.25 | 96.35 | 96.45 | 96.65 |
| dispersion | | | | | | |
| medium for | | | | | | |
| dilution | | | | | | |

| | | | | | |
|---|---|---|---|---|---|
| Microemulsion phase- | 10 to 21 | 14 to 27 | 22 to 41 | 30 to 50 | 39 to 92 |
| forming temperature (° C.) | | | | | |
| Average particle size (nm) | 58 | 73 | 87 | 110 | 137 |

### Examples 10 to 13

Cosmetic compositions of Examples 10 to 13 were each prepared in the same manner as in Example 9, except that the amounts of dimethylpolysiloxane, hydrogenated polydecene, and ion exchanged water constituting the remainder of the dispersion medium used for dilution were changed as indicated in Table 4, and that the microemulsion phase-forming temperature was changed to 20° C. in Example 10, 25° C. in Example 11, or 40° C. in Examples 12 and 13.

### (Results)

As apparent from Table 4, it was found that not only the average particle size of oil droplets but also the microemulsion phase-forming temperature can be adjusted by adjusting the amount of the oil components.

### <Effects of Alkylene Oxide Derivative>

The effects of an alkylene oxide derivative on cosmetic compositions were examined based on Examples 2, 9, 14 and 15.

TABLE 5

| | | Example 9 | Example 14 | Example 2 | Example 15 |
|---|---|---|---|---|---|
| Microemulsion phase | Carboxydecyltrisiloxane | 0.2 | 0.2 | 0.2 | 0.2 |
| | Isostearyl alcohol | 0.3 | 0.3 | 0.3 | 0.3 |
| | PEG-20 glyceryl isostearate (HLB = 13) | 0.4 | 0.4 | 0.4 | 0.4 |
| | Dimethylpolysiloxane | 0.35 | 0.35 | 0.35 | 0.35 |
| | Hydrogenated polydecene | 0.35 | 0.35 | 0.35 | 0.35 |
| | Dipropylene glycol | 1 | 0.8 | 0.6 | 0.4 |
| | POE(14) POP(7) dimethyl ether | 0 | 0.2 | 0.4 | 0.6 |
| | Ion exchanged water | 1 | 1 | 1 | 1 |
| | Aminomethyl propanediol | 0.05 | 0.05 | 0.05 | 0.05 |
| Remainder of dispersion | Ion exchanged water | 96.35 | 96.35 | 96.35 | 96.35 |
| medium for dilution | | | | | |

| | | | | |
|---|---|---|---|---|
| Microemulsion phase-forming temperature (° C.) | 22 to 41 | 24 to 42 | 27 to 43 | 30 to 49 |
| Average particle size (nm) | 87 | 92 | 83 | 126 |

### Examples 14 and 15

Cosmetic compositions of Examples 14 and 15 were each prepared in the same manner as in Example 2, except that the amount of dipropylene glycol and that of POE(14) POP(7) dimethyl ether were changed as indicated in Table 5.

### (Results)

As apparent from Table 5, it was confirmed that a fine emulsion cosmetic composition can be prepared even when an alkylene oxide derivative, such as POE(14) POP(7) dimethyl ether, is used in combination with dipropylene glycol that is a divalent glycol.

### <Effects of Amounts of Oil Components and Alkylene Oxide Derivative>

The effects of the amounts of oil components and an alkylene oxide derivative on cosmetic compositions were examined based on Examples 16 to 20.

TABLE 6

| | | Example 16 | Example 17 | Example 18 | Example 19 | Example 20 |
|---|---|---|---|---|---|---|
| Microemulsion | Carboxydecyltrisiloxane | 0.2 | 0.2 | 0.2 | 0.2 | 0.2 |
| phase | Isostearyl alcohol | 0.3 | 0.3 | 0.3 | 0.3 | 0.3 |
| | PEG-20 glyceryl | 0.4 | 0.4 | 0.4 | 0.4 | 0.4 |
| | isostearate (HLB = 13) | | | | | |
| | Dimethylpolysiloxane | 0.3 | 0.3 | 0.3 | 0.3 | 0.3 |
| | Hydrogenated | 0.3 | 0.3 | 0.3 | 0.3 | 0.3 |
| | polydecene | | | | | |
| | Dipropylene glycol | 1 | 0.8 | 0.7 | 0.6 | 0.4 |
| | POE(14) POP(7) | 0 | 0.2 | 0.3 | 0.4 | 0.6 |
| | dimethyl ether | | | | | |
| | Ion exchanged water | 1 | 1 | 1 | 1 | 1 |
| | Aminomethyl | 0.05 | 0.05 | 0.05 | 0.05 | 0.05 |
| | propanediol | | | | | |
| Remainder of | Ion exchanged water | 96.45 | 96.45 | 96.45 | 96.45 | 96.45 |
| dispersion | | | | | | |
| medium for | | | | | | |
| dilution | | | | | | |

| | | | | | |
|---|---|---|---|---|---|
| Microemulsion phase- | 30 to 50 | 32 to 51 | 32 to 53 | 32 to 54 | 29 to 56 |
| forming temperature (° C.) | | | | | |
| Average particle size (nm) | 110 | 96 | 87 | 101 | 129 |

### Examples 16 to 20

Cosmetic compositions of Examples 16 to 20 were each prepared in the same manner as in Example 2, except that the amount of dimethylpolysiloxane and that of hydrogenated polydecene were changed as indicated in Table 6, that the amount of dipropylene glycol and that of an alkylene oxide derivative such as POE(14) POP(7) dimethyl ether were changed in Examples 16 to 18 and 20 as indicated in Table 6, and that the microemulsion phase-forming temperature was changed to 40° C. in Examples 17 to 19.

### (Results)

As apparent from Table 6, it was confirmed that, even when the amounts of the oil components were further reduced as compared to the embodiments indicated in Table 5, a fine emulsion cosmetic composition can be prepared in a system where dipropylene glycol and POE(14) POP(7) dimethyl ether are used in combination.

<Effects of Divalent Glycol Other than Dipropylene Glycol>

The effects of a divalent glycol other than dipropylene glycol on cosmetic compositions were examined based on Examples 9 and 21 to 24.

TABLE 7

| | | Example 9 | Example 21 | Example 22 | Example 23 | Example 24 |
|---|---|---|---|---|---|---|
| Microemulsion | Carboxydecyltrisiloxane | 0.2 | 0.2 | 0.2 | 0.2 | 0.2 |
| phase | Isostearyl alcohol | 0.3 | 0.3 | 0.3 | 0.3 | 0.3 |
| | PEG-20 glyceryl | 0.4 | 0.4 | 0.4 | 0.4 | 0.4 |
| | isostearate (HLB = 13) | | | | | |
| | Dimethylpolysiloxane | 0.35 | 0.35 | 0.35 | 0.35 | 0.35 |
| | Hydrogenated | 0.35 | 0.35 | 0.35 | 0.35 | 0.35 |
| | polydecene | | | | | |
| | Dipropylene glycol | 1 | 0.8 | 0.6 | 0.2 | 0 |
| | 1,3-butylene glycol | 0 | 0.2 | 0.4 | 0.8 | 1 |
| | Ion exchanged water | 1 | 1 | 1 | 1 | 1 |
| | Aminomethyl | 0.05 | 0.05 | 0.05 | 0.05 | 0.05 |
| | propanediol | | | | | |
| Remainder of | Ion exchanged water | 96.35 | 96.35 | 96.35 | 96.35 | 96.35 |
| dispersion | | | | | | |
| medium for | | | | | | |
| dilution | | | | | | |

| | | | | | |
|---|---|---|---|---|---|
| Microemulsion phase- | 22 to 41 | 23 to 43 | 37 to 51 | 52 to 69 | 61 to 73 |
| forming temperature (° C.) | | | | | |
| Average particle size (nm) | 87 | 92 | 97 | 103 | 98 |

### Examples 21 to 24

Cosmetic compositions of Examples 21 to 24 were each prepared in the same manner as in Example 9, except that the blending ratio of dipropylene glycol and 1,3-butylene glycol was changed as indicated in Table 7, and that the microemulsion phase-forming temperature was changed to 40° C. in Example 22, 55° C. in Example 23, or 65° C. in Example 24.

### (Results)

As apparent from Table 7, it was confirmed that a fine emulsion cosmetic composition can be prepared even in a system where other divalent glycol such as 1,3-butylene glycol, not limited to dipropylene glycol, is used. In addition, it was found that the microemulsion phase-forming temperature can be further lowered in a system where dipropylene glycol is used as a divalent glycol.

### <Effects of Blending Ratio of Water and Divalent Glycol Used for Preparation of Microemulsion Phase>

The effects of the blending ratio of water and divalent glycol that are used for the preparation of a microemulsion phase on cosmetic compositions were examined based on Examples 9 and 25 to 27.

TABLE 8

| | | Example 25 | Example 26 | Example 9 | Example 27 |
|---|---|---|---|---|---|
| Microemulsion phase | Carboxydecyltrisiloxane | 0.2 | 0.2 | 0.2 | 0.2 |
| | Isostearyl alcohol | 0.3 | 0.3 | 0.3 | 0.3 |
| | PEG-20 glyceryl isostearate (HLB = 13) | 0.4 | 0.4 | 0.4 | 0.4 |
| | Dimethylpolysiloxane | 0.35 | 0.35 | 0.35 | 0.35 |
| | Hydrogenated polydecene | 0.35 | 0.35 | 0.35 | 0.35 |
| | Dipropylene glycol | 0.8 | 0.9 | 1 | 1.1 |
| | Ion exchanged water | 1.2 | 1.1 | 1 | 0.9 |
| | Aminomethyl propanediol | 0.05 | 0.05 | 0.05 | 0.05 |
| Remainder of dispersion | Ion exchanged water | 96.35 | 96.35 | 96.35 | 96.35 |
| medium for dilution | | | | | |

| | | | | |
|---|---|---|---|---|
| Microemulsion phase-forming temperature (° C.) | 49 to 65 | 35 to 54 | 22 to 41 | ≤19¹⁾ |
| Average particle size (nm) | 97 | 95 | 87 | 91 |

¹⁾It was confirmed that a microemulsion phase could be formed at a temperature of as low as about 0° C.; however, the lower limit value of the microemulsion phase-forming temperature could not be measured.

### Examples 25 to 27

Cosmetic compositions of Examples 25 to 27 were each prepared in the same manner as in Example 9, except that the blending ratio of dipropylene glycol and ion exchanged water used for the preparation of a microemulsion phase was changed as indicated in Table 8, and that the microemulsion phase-forming temperature was changed to 50° C. in Example 25, 40° C. in Example 26, or 15° C. in Example 27.

### (Results)

As apparent from Table 8, it was found that, by adjusting the blending ratio of a divalent glycol and water used for the preparation of a microemulsion phase, the microemulsion phase-forming temperature can be adjusted without a large change in the average particle size of oil droplets.

### <Effects of Lower Alcohol in Remainder of Dispersion Medium for Dilution>

The effects of a lower alcohol contained in the remainder of the dispersion medium for dilution on cosmetic compositions were examined based on Examples 2 and 28 to 30 as well as Comparative Examples 7 and 8. In Table 9, “EtOH” denotes ethanol and, for example, “EtOH 10%” denotes that, in the remainder of a dispersion medium that contained water and ethanol and used for dilution, the content of ethanol was 10% by mass.

TABLE 9

| | | | | | | Compar- | Compar- |
|---|---|---|---|---|---|---|---|
| | | | | | | ative | ative |
| | | Example 2 | Example 28 | Example 29 | Example 30 | Example 7 | Example 8 |
| Microemulsion | Carboxydecyltrisiloxane | 0.2 | 0.2 | 0.2 | 0.2 | 0.2 | 0.2 |
| phase | Isostearyl alcohol | 0.3 | 0.3 | 0.3 | 0.3 | 0.3 | 0.3 |
| | PEG-20 glyceryl isostearate (HLB = 13) | 0.4 | 0.4 | 0.4 | 0.4 | 0.4 | 0.4 |
| | Dimethylpolysiloxane | 0.35 | 0.35 | 0.35 | 0.35 | 0.35 | 0.35 |
| | Hydrogenated polydecene | 0.35 | 0.35 | 0.35 | 0.35 | 0.35 | 0.35 |
| | Dipropylene glycol | 0.6 | 0.6 | 0.6 | 0.6 | 0.6 | 0.6 |
| | POE(14) POP(7) dimethyl ether | 0.4 | 0.4 | 0.4 | 0.4 | 0.4 | 0.4 |
| | Ion exchanged water | 1 | 1 | 1 | 1 | 1 | 1 |
| | Aminomethyl propanediol | 0.05 | 0.05 | 0.05 | 0.05 | 0.05 | 0.05 |

| | | | | | | |
|---|---|---|---|---|---|---|
| Amount of remainder | 96.35 | 96.35 | 96.35 | 96.35 | 96.35 | 96.35 |
| of dispersion medium for dilution | | | | | | |
| Type of remainder of | water | EtOH 10% | EtOH 20% | EtOH 30% | EtOH 40% | EtOH 50% |
| dispersion medium for dilution | | | | | | |
| Average particle size (nm) | 83 | 49 | 18 | 62 | 167 | 239 |

### Examples 28 to 30 and Comparative Examples 7 and 8

Cosmetic composition of Examples 28 to 30 and Comparative Examples 7 and 8 were each prepared in the same manner as in Example 2, except that the type of the remainder of dispersion medium for dilution was changed as indicated in Table 9.

### (Results)

As apparent from the results of Table 9, it was confirmed that the average particle size of oil droplets in a cosmetic composition can be greatly reduced by using a dispersion medium for dilution that contains a prescribed amount of ethanol in addition to water. It is believed easy to infer from the results that such an effect of reducing the average particle size can also be exerted in the same manner when a lower alcohol other than ethanol that exhibits performance similar to that of ethanol, such as a monohydric alcohol containing an alkyl group having 1 to 5 carbon, is used.

### FORMULATION EXAMPLES OF FINE EMULSION COSMETIC COMPOSITION

Formulation examples of the fine emulsion cosmetic composition of the present invention include, but not limited to, the followings.

### Formulation Example 1: Transparent Skin Lotion

| (Components) | (% by mass) |
|---|---|
| Purified water | remainder |
| EDTA-2Na•2H₂O | 0.03 |
| Glycerin | 5.0 |
| Dipropylene glycol | 5.0 |
| Polyethylene glycol 300 | 3.0 |
| Ethanol | 5.0 |
| Phenoxyethanol | 0.5 |
| Carboxydecyltrisiloxane | 0.2 |
| Isostearyl alcohol | 0.3 |
| PEG-20 glyceryl isostearate | 0.4 |
| Hydrogenated polydecene | 0.35 |
| Dimethicone | 0.35 |
| POE(14) POP(7) dimethyl ether | 0.4 |
| Aminomethyl propanediol | 0.05 |

### (Method of Producing Transparent Skin Lotion)

A portion of purified water, EDTA-2Na.2H₂O, glycerin, a portion of dipropylene glycol, polyethylene glycol 300, a portion of ethanol, and phenoxyethanol were dissolved by mixing at 25° C. to prepare a mixture 1. Subsequently, a portion of purified water and a portion of ethanol were mixed at 25° C. to prepare a mixture 2. Then, carboxydecyltrisiloxane, isostearyl alcohol, PEG-20 glyceryl isostearate, hydrogenated polydecene, dimethicone, POE(14) POP(7) dimethyl ether, aminomethyl propanediol, a portion of purified water, and a portion of dipropylene glycol were dissolved by mixing at 40° C. to prepare a microemulsion. The thus obtained microemulsion was added to the mixture 2, and the resultant was mixed with stirring to dilute the microemulsion, whereby an emulsion was prepared. Further, this emulsion was added to the mixture 1, and the resultant was mixed with stirring to produce a transparent skin lotion. Oil droplets contained in the thus obtained skin lotion had an average particle size of 40 nm, and the skin lotion had a pH of 8.35 and an L value of 95.

### Formulation Example 2: Beauty Lotion

| (Components) | (% by mass) |
|---|---|
| Purified water | remainder |
| EDTA-2Na•2H₂O | 0.03 |
| Glycerin | 5.0 |
| Dipropylene glycol | 5.0 |
| Polyethylene glycol 300 | 3.0 |
| Ethanol | 5.0 |
| Phenoxyethanol | 0.5 |
| Stearoxyhydroxypropylmethyl cellulose | 0.2 |
| Carboxydecyltrisiloxane | 0.2 |
| Isostearyl alcohol | 0.3 |
| PEG-20 glyceryl isostearate | 0.4 |
| Hydrogenated polydecene | 0.35 |
| Dimethicone | 0.35 |
| POE(14) POP(7) dimethyl ether | 0.4 |
| Aminomethyl propanediol | 0.05 |

### (Method of Producing Beauty Lotion)

A portion of purified water, EDTA-2Na.2H₂O, glycerin, a portion of dipropylene glycol, polyethylene glycol 300, a portion of ethanol, phenoxyethanol, and stearoxyhydroxypropylmethyl cellulose were dissolved by mixing at 75° C. to prepare a mixture 3. Subsequently, a portion of purified water and a portion of ethanol were mixed at 25° C. to prepare a mixture 4. Then, carboxydecyltrisiloxane, isostearyl alcohol, PEG-20 glyceryl isostearate, hydrogenated polydecene, dimethicone, POE(14) POP(7) dimethyl ether, aminomethyl propanediol, a portion of purified water, and a portion of dipropylene glycol were dissolved by mixing at 40° C. to prepare a microemulsion. The thus obtained microemulsion was added to the mixture 4, and the resultant was mixed with stirring to dilute the microemulsion, whereby an emulsion was prepared. Further, this emulsion was added to the mixture 3, and the resultant was mixed with stirring to produce a beauty lotion. The thus obtained beauty lotion had a viscosity of 770 mPa/s as measured by a B-type viscometer (rotor No. 2, 12 rpm), a pH of 8.25, and an L value of 90.

1. An oil-in-water fine emulsion cosmetic composition, containing:
a dispersion medium that contains water and a divalent glycol;
an oil component that is dispersed in the dispersion medium and contains a silicone oil and a hydrocarbon oil;
a carboxy-modified silicone-based surfactant represented by the following Formula 1;
at least one selected from a higher alcohol and a higher fatty acid that are liquid at 25° C. and have 16 to 22 carbon atoms; and
a nonionic surfactant that contains a polyoxyethylene chain and has an HLB of 12 to 17,
the cosmetic composition having oil droplets of an average particle size of 150 nm or smaller: 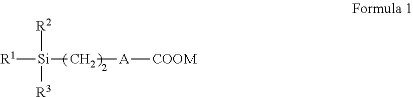
wherein,
at least one of R¹ to R³ is a substituent represented by —O—Si(R⁴)₃ wherein R⁴ is at least one selected from an alkyl group having 1 to 6 carbon atoms and a phenyl group and, when any one of R¹ to R³ is not the substituent, the others are substituted or unsubstituted monovalent hydrocarbon groups that are optionally the same or different;
A is a linear or branched alkylene group represented by C_{q}H_{2q} wherein q is an integer of 0 to 20, and
M is a metal atom or an organic cation.

2. The cosmetic composition according to claim 1, wherein the carboxy-modified silicone-based surfactant, the at least one selected from the higher alcohol and the higher fatty acid, and the nonionic surfactant form liquid interfacial films of the oil droplets in a 25° C. atmosphere.

3. The cosmetic composition according to claim 1, wherein the higher alcohol is at least one selected from isostearyl alcohol and oleyl alcohol, and the higher fatty acid is at least one selected from isostearic acid and oleic acid.

4. The cosmetic composition according to claim 1, wherein the silicone oil and the hydrocarbon oil are contained in the oil component in an amount of 82% by mass or more.

5. The cosmetic composition according to claim 1, wherein a mass ratio of the silicone oil and the hydrocarbon oil is 1:9 to 9:1.

6. The cosmetic composition according to claim 1, wherein a mass ratio of the oil component is 0.45 to 1.0 with respect to a total amount of the carboxy-modified silicone-based surfactant, the at least one selected from the higher alcohol and the higher fatty acid, and the nonionic surfactant.

7. The cosmetic composition according to claim 1, wherein the oil droplets have an average particle size of 55 nm or smaller and contain a low alcohol.

8. A method of producing the cosmetic composition according to claim 1, the method including:
preparing a microemulsion by mixing a portion of the dispersion medium, the oil component, the carboxy-modified silicone-based surfactant, the at least one selected from the higher alcohol and the higher fatty acid, and the nonionic surfactant; and
diluting the microemulsion with an addition of a remainder of the dispersion medium, or diluting a remainder of the dispersion medium with an addition of the microemulsion.

9. The method according to claim 8, wherein the remainder of the dispersion medium contains a lower alcohol.
